(19) United States
(12) Patent Application Publication
(10) Pub. No.: US 2016/0271119 A1
(43) Pub. Date: Sep. 22, 2016

(54) Method of Treating Periodontal Disease by Altering an Epithelial to Mesenchymal Transition

(71) Applicant: Hans M. Albertsen, Salt Lake City, UT (US)

(72) Inventor: Hans M. Albertsen, Salt Lake City, UT (US)

(21) Appl. No.: 15/168,033

(22) Filed: May 28, 2016

Related U.S. Application Data
(63) Continuation-in-part of application No. 15/141,964, filed on Apr. 29, 2016.
(60) Provisional application No. 62/170,346, filed on Jun. 3, 2015.
(60) Provisional application No. 62/247,847, filed on Oct. 29, 2015.

(51) Int. Cl. A61K 31/44; A61K 31/122; A61K 31/12; A61K 31/05; A61K 9/00; A61K 31/65

(52) U.S. Cl. CPC A61K 31/44; A61K 9/0019; A61K 9/0063; A61K 31/65; A61K 31/12; A61K 31/05; A61K 31/122

(57) ABSTRACT

The present invention is a method of treating periodontal disease and in particular a method of treating gingivitis and periodontitis by administering an Epithelial-to-mesenchymal transition altering compound. The compound may be a natural compound or a synthetic compound. The compound may be in pill form, liquid form (.e.g. mouthwash), cream form (e.g. a topical cream), ointment form, paste form (e.g. toothpaste), lotion form, gel form (e.g. toothgel), a powder form, or sheet form (e.g. a sheet adapted to be inserted into a periodontal pocket and to dissolve thereafter).

### CROSS REFERENCE TO RELATED APPLICATIONS

This nonprovisional utility patent application is a continuation-in-part of and claims the benefit under 35 USC §120 to co-pending U.S. application Ser. No. 15/141,964 filed Apr. 29, 2016, which claims the benefit under 35 USC §119(e) of U.S. provisional application No. 62/170,346 filed Jun. 3, 2015, and this application claims the benefit under 35 USC §119(e) of U.S. provisional application No. 62/247,847 filed Oct. 29, 2015, all of which are incorporated in their entirety by this reference.

### FIELD OF THE INVENTION

The present invention relates to methods for preventing or treating periodontal disease (PD) by affecting the cellular state of the periodontal epithelium.

### BACKGROUND OF THE INVENTION

For the purposes of this application, PD shall be understood to mean at least one of gingivitis (GV) and periodontitis (PS) (see Appx A). GV is a non-destructive and milder form of PD that may occur in response to bacterial biofilms (also called plaque) adherent to tooth surfaces (see Appx B). PS is a more severe form of PD in which the pocket around a tooth becomes infected and where the resulting inflammation causes the gum to recede, leading to alveolar bone resorption, and ultimately may lead to tooth loss (see Appx C). Chronic PS is a chronic inflammatory disease that affects the soft and hard structures that support the teeth. It is caused by bacterial infection and inflammation that destroys the attachment fibers and supporting bones that hold the teeth in the mouth (see Appx D).

PD affects approximately half of American adults and severity correlates with age (Eke, et al. (2012), Prevalence of periodontitis in adults in the United States: 2009 and 2010. J Dent Res. 91:914-920). It is believed that bacterial toxins and the body's natural response to infection start to break down the bone and connective tissue that hold teeth in place. If not treated, the bones, gums, and tissue that support the teeth are destroyed. It is known that poor oral hygiene contributes to the risk for developing PD. It is also known that age and certain life-style factors like smoking contribute to PD-risk. Further, it has been shown that hereditary (genetic) factors contribute to the risk for developing PD and that PD can be classified as a complex disease. In many cases, PD can be prevented by good oral hygiene. Traditionally, treatment of PD has included antibiotic treatment of the bacterial infection and anti-inflammatory treatments. An exemplary prior art method of treating PD is disclosed in U.S. Pat. No. 8,865,198 to Avramoff et al. which is incorporated herein in its entirety by this reference. In more severe cases of PD, PD may also be treated surgically.

The epithelial sheet that covers soft tissue surfaces of the oral cavity provides a protective barrier to mechanical and microbial insults. Epithelial tissue in the periodontal areas is defined as gingival epithelium, sulcular epithelium, and junctional epithelium. In particular the sulcular and junctional epithelia are vulnerable to bacterial infections that lead to PD (see Appx E). Maintenance of the epithelial barrier integrity is essential to good oral health and to the prevention bacterial infections.

Epithelial repair (wound-healing) involves the trans-differentiation of epithelial cells into motile mesenchymal cells in a process known as epithelial-mesenchymal transition (EMT) (see Appx F). During this process the epithelial barrier integrity is temporarily lost during the repair. Under normal circumstances the epithelial barrier integrity is re-established by the reverse process known as mesenchymal-epithelial transition (MET) (Lamouille, et al. (2014), Molecular mechanisms of epithelial-mesenchymal transition. Nat Rev Mol Cell Biol. 15:178-196). However, pathogenic microbes can take advantage of this temporary vulnerability to infect subjacent tissues. EMT is integral in embryonic development, wound healing, and stem cell behavior, but it also contributes pathologically to fibrosis and cancer progression (Barriere, et al. (2015), Epithelial Mesenchymal Transition: a double-edged sword. Clin Transl Med. 4:14). It has become evident that bacterial pathogens can employ different strategies to invade and infect epithelial tissues and their underlying structures including the direct manipulation to induce the EMT process and to prevent MET (Barreau and Hugot (2014), Intestinal barrier dysfunction triggered by invasive bacteria. Curr Opin Microbiol. 17:91-98; Zihni, et al. (2014), Signalling at tight junctions during epithelial differentiation and microbial pathogenesis. J Cell Sci. 127:3401-3413; Hess and Rambukkana (2015), Bacterial-induced cell reprogramming to stem cell-like cells: new premise in host-pathogen interactions. Curr Opin Microbiol. 23:179-188; Ribet and Cossart (2015), How bacterial pathogens colonize their hosts and invade deeper tissues. Microbes Infect. 17:173-183; Leone, et al. (2016), Klebsiella pneumoniae Is Able to Trigger Epithelial-Mesenchymal Transition Process in Cultured Airway Epithelial Cells. PLoS One. 11:e0146365).

### SUMMARY OF THE INVENTION

The present invention is a method of treating PD (including the prevention of PD, the prevention of progress of PD, and the reversal of PD, and the elimination of PD), by altering EMT in a patient having or being predisposed to PD. EMT of a patient is preferably altered by administering an EMT altering composition (EAC). The EAC specifically preferably affects cellular remodeling of epithelial membranes to prevent microbial pathogens from infecting periodontal pockets or to reverse established PD by restoring healthy epithelial membranes in the periodontal pocket. More specifically, the method comprises the administration of an EAC that can prevent or reverse the EMT mechanism triggered by oral pathogens during the development and progression of periodontal disease. The EAC also affects the EMT repair process and/or the MET re-epithelization process so as to prevent and treat PD. The EAC can be administered either locally or systemically. The EAC can be administered alone, in conjunction with, or co-formulated with therapeutically effective amounts of at least one antibiotic agent and at least one anti-inflammatory agent, or a combination of such agents.

The treatment disclosed herein is specifically intended to inhibit or reverse EMT—a prerequisite for developing PD. Depending on the degree of PD present in a patient, or the genetic and environmental risk to which a patient is exposed, a graded range of treatment is anticipated. Younger, asymptomatic patients may be treated by simple over-the-counter supplements, like a toothpaste or mouthwash formulated to include an EAC, to maintain good epithelial health and thus reduce the risk of developing PD. Older and/or more severely affected patients may be treated by compositions available only by prescription and by specialized care administered by their oral care provider. As all individuals have a unique genetic make-up and live in unique environments the EAC formulations may benefit from optimization of the formulations and dosages in a manner dependent on genetic and environmental factors specific to a given patient.

By way of example, the goals of administering an EAC include prevention of PD, control of symptoms of PD, regression of PD lesions, reduction in secondary complications, reduction in pain, reduction in PD-related inflammation, reduction of alveolar bone-loss, and PD-related tooth loss. Moreover, the treatment of a PD asymptomatic patient with an EAC to prevent PD onset or development wherein such PD asymptomatic patient is found to have at least one PD associated life-style, age-related, or genetic risk factor (including having a genetic biomarker associated with existence or predisposition of PD) is also contemplated. A set of markers characteristic of the epithelial state include the expression of E-cadherin (CDH1), whereas the expression of N-cadherin (CDH2) and Vimentin (VIM) are characteristic of the mesenchymal state. The ratio of expression between, for example, CDH1 and CDH2 can therefore be used to assess the cellular state of an epithelial tissue sample as epithelial or mesenchymal. For instance, if the ratio of CDH1/CDH2, such as observed in a periodontal pocket biopsy sample of a PD asymptomatic patient, is less than 1.0, the ratio is considered to be a marker associated with a majority mesenchymal state and an increased risk of PD. Thus in response to a patient having a CDH1/CDH2 ratio of less than 1.0, an EAC would be administered to the patient. Moreover, it is contemplated that the degree of CDH1/CDH2 ratio observed may correlate to the amount of EAC administered, under the protocol of the lower the CDH1/CDH2 ratio observed, the greater the amount of EAC administered. Conversely, if the ratio of CDH1/CDH2, such as observed in a periodontal pocket biopsy sample of a PD asymptomatic patient, is greater than 1.0, the ratio is considered to be a marker associated with a majority epithelial state and a decreased risk of PD. Thus in response to a patient having a CDH1/CDH2 ratio of greater than 1.0, an EAC would not be administered to the patient. Other ratio markers are also contemplated. For instance the ratio of CDH1/VIM may function as an alternate or substitute ratio for the ratio of CDH1/CDH2. EMT inhibitors include a range of known compounds that have been tested in for efficacy in tissue-culture based experiments by measuring, for example, the ratio between E-cadherin and N-cadherin. Biologic experiments employing gene expression measurements of this kind are used to assess pharmaceutical compounds for their ability to inhibit EMT and include for instance compounds like rapamycin, 17-AAG and LY294002, and A83-01 and GDC-0941 (Reka, et al. (2011), Identifying inhibitors of epithelial-mesenchymal transition by connectivity map-based systems approach. J Thorac Oncol. 6:1784-1792; Chua, et al. (2012), A cell-based small molecule screening method for identifying inhibitors of epithelial-mesenchymal transition in carcinoma. PLoS One. 7:e33183). It is expected that the range of EAC compounds will grow and that new members of this group will also prove useful in the treatment of PD via their effect on EMT.

### DETAILED DESCRIPTION OF THE INVENTION

The invention disclosed herein is a method of preventing or treating PD in a patient by administering to the patient an EAC in an amount effective to alter EMT in the patient. Included in such EMT alteration are EMT prevention, EMT inhibition, EMT reduction, and EMT reversal. Possible EACs include both natural and synthetic EACs. EACs may also be a specific microRNA, a siRNA and an antibody. Specific exemplary EACs include, Sorafenib, Metacycline, Diphenyl difluoroketone, Resveratrol, Curcumin, Emodin, and others (Subramaniam, et al. (2008), Diphenyl difluoroketone: a curcumin derivative with potent in vivo anticancer activity. Cancer Res. 68:1962-1969; Hu, et al. (2010), Curcumin inhibits transforming growth factor-beta activity via inhibition of Smad signaling in HK-2 cells. Am J Nephrol. 31:332-341; Singh, et al. (2011), Resveratrol modulates the expression of PTGS2 and cellular proliferation in the normal rat endometrium in an AKT-dependent manner. Biol Reprod. 84:1045-1052; Dandawate, et al. (2013), Novel strategies targeting cancer stem cells through phytochemicals and their analogs. Drug Deliv Transl Res. 3:165-182; Li, et al. (2013), Curcumin inhibits transforming growth factor-beta1-induced EMT via PPARgamma pathway, not Smad pathway in renal tubular epithelial cells. PLoS One. 8:e58848; Li, et al. (2013), Resveratrol inhibits the epithelial-mesenchymal transition of pancreatic cancer cells via suppression of the PI-3K/Akt/NF-kappaB pathway. Curr Med Chem. 20:4185-4194; Rico-Leo, et al. (2013), Dioxin receptor expression inhibits basal and transforming growth factor beta-induced epithelial-to-mesenchymal transition. J Biol Chem. 288:7841-7856; Diaz-Lopez, et al. (2014), Role of microRNA in epithelial to mesenchymal transition and metastasis and clinical perspectives. Cancer Manag Res. 6:205-216; Montgomery, et al. (2014), MicroRNA mimicry blocks pulmonary fibrosis. EMBO Mol Med. 6:1347-1356; Xi, et al. (2014), Inhibition of epithelial-to-mesenchymal transition and pulmonary fibrosis by methacycline. Am J Respir Cell Mol Biol. 50:51-60; Ji, et al. (2015), Resveratrol suppresses epithelial-to-mesenchymal transition in colorectal cancer through TGF-beta1/Smads signaling pathway mediated Snail/E-cadherin expression. BMC Cancer. 15:97; Jia, et al. (2015), Sorafenib ameliorates renal fibrosis through inhibition of TGF-beta-induced epithelial-mesenchymal transition. PLoS One. 10:e0117757; Liu, et al. (2015), miR-221 facilitates the TGFbeta1-induced epithelial-mesenchymal transition in human bladder cancer cells by targeting STMN1. BMC Urol. 15:36; Parvani, et al. (2015), Silencing beta3 Integrin by Targeted ECO/siRNA Nanoparticles Inhibits EMT and Metastasis of Triple-Negative Breast Cancer. Cancer Res; Thacker and Karunagaran (2015), Curcumin and Emodin Down-Regulate TGF-beta Signaling Pathway in Human Cervical Cancer Cells. PLoS One. 10:e0120045; Zaravinos (2015), The Regulatory Role of MicroRNAs in EMT and Cancer. J Oncol. 2015:865816; Zhang, et al. (2015), Proton pump inhibitor pantoprazole abrogates adriamycin-resistant gastric cancer cell invasiveness via suppression of Akt/GSK-beta/beta-catenin signaling and epithelial-mesenchymal transition. Cancer Lett. 356:704-712; Zhang, et al. (2015), MicroRNA-33b, upregulated by EF24, a curcumin analog, suppresses the epithelial-to-mesenchymal transition (EMT) and migratory potential of melanoma cells by targeting HMGA2. Toxicol Lett. 234:151-161). Methods of administration of such EACs include oral consumption, inhaled, transdermal absorption, intravenous, and subcutaneous or intra-muscular injection. In the case of a patient manifesting symptoms of PD, the EAC may be for instance administered to the patient without further analysis of the patient. In the case of a PD asymptomatic patient, the EAC may be for instance administered to the patient after having determined that the patient is exposed to at least one PD associated life-style factor, age-related factor, environmental factor, or a genetic factor (marker) present in the genetic material of the patient.

The disclosed EAC may be administered either as a single compound or as a combination of multiple EACs. An EAC might also be administered in combination with, for example, a nonsteroidal anti-inflammatory drug (NSAID) or in combination with, for example, an antibiotic. Further, the EAC may form a component of a pill, a liquid (.e.g. mouthwash), a cream (e.g. a topical cream), an ointment, a paste (e.g. toothpaste), a lotion, a gel (e.g. toothgel), a delayed release powder, or a sheet (e.g. a sheet adapted to be inserted into a periodontal pocket and to dissolve over time).

In a first exemplary embodiment of the invention, the invention comprises the step of administering to a patient known to have PD or to manifest symptoms of PD, an oral administration of an EAC of Sorafenib in pill form, resulting in the reversal of EMT in the patient and resulting in the reduction of PD symptoms in the patient.

In a second exemplary embodiment of the invention, the invention comprises the step of administering at recurrent intervals an oral application of Metacycline in cream form having an EAC therein to a patient being asymptomatic of PD but known to be a smoker (a known risk-factor for PD) so as to inhibit EMT in the patient so as to result in prevention of PD symptoms in the patient.

In a third exemplary embodiment of the invention, the invention comprises the step of periodically administering to the mouth of a patient applications of at least one of a toothpaste, a mouthwash, and a sheet, wherein the toothpaste, mouthwash, and sheet each include an EAC as a component thereof. It is noted that the application of the toothpaste, mouthwash, and sheet may be self-administered or administered by another such as a dental care provider.

In addition to reducing or preventing the symptoms of PD, a benefit of the disclosed invention is that the disclosed treatment of PD avoids surgical methods of treatment which is a traditional method of treating PS.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of treating PD in a subject comprising administering an EAC to said subject such that EMT of said subject is altered.

2. The method of claim 1, wherein said PD defines at least one of GV and PS.

3. The method of claim 1, wherein said EAC defines at least one of a natural EAC and a synthetic EAC.

4. The method of claim 1, wherein said EAC defines at least one of Sorafenib, Metacycline, Diphenyl difluoroketone, Resveratrol, Curcumin, and Emodin.

5. The method of claim 1, wherein said EAC defines an EAC in at least one of a pill form, a liquid form, a cream form, an ointment form, a paste form, a lotion form, a gel form, a powder form, and a sheet form.

6. The method of claim 5, wherein said liquid form defines a mouthwash form, and wherein said cream form defines a topical cream form, and wherein said paste form defines a toothpaste form, and wherein said gel form defines a toothgel, and wherein said sheet form defines a sheet form adapted to be inserted into a periodontal pocket.

7. The method of claim 1, wherein said administration further defines injecting said EAC into said subject, implanting said EAC into said subject, and applying said EAC in a sheet form into the periodontal pocket of said subject.

8. A method of preventing PD in a PD asymptomatic subject by maintaining epithelial barrier integrity comprising administering an EAC to said subject such that EMT of said subject is altered.

9. The method of claim 8, wherein said PD defines at least one of GV and PS.

10. The method of claim 8, wherein said EAC defines at least one of a natural EAC and a synthetic EAC.

11. The method of claim 8, wherein said EAC defines at least one of Sorafenib, Metacycline, Diphenyl difluoroketone, Resveratrol, Curcumin, and Emodin.

12. The method of claim 8, wherein said EAC defines an EAC in at least one of a pill form, a liquid form, a cream form, an ointment form, a paste form, a lotion form, a gel form, a powder form, and a sheet form.

13. The method of claim 12, wherein said liquid form defines a mouthwash form, and wherein said cream form defines a topical cream form, and wherein said paste form defines a toothpaste form, and wherein said gel form defines a toothgel, and wherein said sheet form defines a sheet form adapted to be inserted into a periodontal pocket.

14. The method of claim 8, wherein said administration further defines injecting said EAC into said subject, implanting said EAC into said subject, and applying said EAC in a sheet form into the periodontal pocket of said subject.

15. A method of treating PD in a PD asymptomatic subject known to have at least one PD increased risk associated biomarker in said subject comprising administering an EAC to said subject such that EMT of said subject is altered.

16. The method of claim 15, wherein said at least one PD increased risk associated biomarker defines a CDH1/CDH2 expression ration of less than 1.0.

17. The method of claim 15, wherein said EAC defines at least one of Sorafenib, Metacycline, Diphenyl difluoroketone, Resveratrol, Curcumin, and Emodin.

18. The method of claim 15, wherein said EAC defines an EAC in at least one of a pill form, a liquid form, a cream form, an ointment form, a paste form, a lotion form, a gel form, a powder form, and a sheet form.

19. The method of claim 18, wherein said liquid form defines a mouthwash form, and wherein said cream form defines a topical cream form, and wherein said paste form defines a toothpaste form, and wherein said gel form defines a toothgel, and wherein said sheet form defines a sheet form adapted to be inserted into a periodontal pocket.

20. The method of claim 15, wherein said administration further defines injecting said EAC into said subject, implanting said EAC into said subject, and applying said EAC in a sheet form into the periodontal pocket of said subject.
